(19) United States
(12) Patent Application Publication
(10) Pub. No.: US 2002/0127740 A1
(43) Pub. Date: Sep. 12, 2002

(54) Quantitative microfluidic biochip and method of use

(76) Inventor: Winston Z. Ho, Hacienda Heights, CA (US)

Correspondence Address: Winston Z. Ho, 14541 Langhill Drive, Hacienda Heights, CA, 91745, US

(21) Appl. No.: 09/973,209

(22) Filed: Oct. 9, 2001

Related U.S. Application Data
(60) Provisional application No. 60/287,781, filed on May 2, 2001.
(60) Provisional application No. 60/273,486, filed on Mar. 6, 2001.

(51) Int. Cl. C12M 1/34; G01N 33/543; G01N 33/553; C12Q 1/68

(52) U.S. Cl. 436/518; 436/526; 435/287.2; 435/6

(57) ABSTRACT

Method is disclosed to provide constant and consistent amount of samples or reagent solutions for performing biological assay in a microfluidic biochip platform. A method and apparatus comprises a plurality of microfluidic channels with constant cross-section area in closed confinement, said microfluidic channels transporting fluids to at least one reaction zone immobilized with biological probes. The fluids in said microfluidic channels is transported to said reaction zone and reacted with the probes. The reaction volume, which is equal to the cross-section area multiply with the length of said channel overlaid on said reaction zone, is therefore a constant number. In addition, a method for immobilizing an array of biological probes in the microfluidic biochip arrangement, which is simple, flexible, and controllable for immobilizing, removing, and replenishing new probes is disclosed.

### RELATED APPLICATION

The present application claims the benefit of 35 U.S.C. 111(b) Provisional applications Serial No. 60/287,781 filed May 2, 2001 entitled “Biochip with high precision in sampling volume”, and No. 60/273,486 filed Mar. 6, 2001 entitled “Bioactive probes immobilization method on biochips”.

### FIELD OF THE INVENTION

The invention is related to a method and biochip apparatus that utilizes microfluidic mechanism to perform biological reactions in closed confinement of a micrometer scale microchannels. The resulting biochip can be used to detect chemical and biological species, for example, cardiac markers, infectious diseases, gene diseases and mutation, cancers, hormones, bacteria, drugs, and many other biological analytes, with high reliability and precision.

### BACKGROUND OF THE INVENTION

The microfluidics-based biochips have broad application in fields of biotechnology, molecular biology, and clinical diagnostics. The biochip offers the possibility to rapidly and easily perform multiple biological and chemical tests in a very small platform. However, as the sampling volume in the biochips being reduced from milliliters to microliters, nanoliters, or picoliters, consistently and accurately delivering the specified small amount of sample or reagents to a micrometer sensing area on the chip becomes very difficult. The practical concerns that in all reality, the detection of analytes is not simply a qualitative some or none, but requires quantitative assay in order to achieve a value for analyte concentration based on a specified sample volume. Thus, the first step in the assay, which is the deposition of sample into the assay system by an operator, for example, 12 μl instead of 10 μl, may in fact already creates 20% variation. There is no guarantee that the operator will deliver the exact amount of sample as specified. Furthermore, even if the specified amount of sample is loaded, it may not be delivered to the reaction zone in the exact amount. In a typical biochip, there is no mechanism to assure that the entire sample finds its way to the reaction area.

For biochip manufacturers, it has been a daunting challenge to deliver the constant volume of either sample or reagents to the reaction area for quantitative assay. In summary, variable reaction volumes in microfluidic-based biochips are commonly generated from:

1. Variation in sample volumes from the operator

2. Variation in reagent volumes in the pre-package microwells or pouches

3. Variation in the residual liquid left in the wells or channels.

4. Air or bubbles generated or trapped in the microfluidic system.

These practical problems are critical for many quantitative clinical diagnostics. The previous solutions to mitigate these problems are to use gates or valves to control and meter exact amount of sample or reagents. Unfortunately, the mechanisms of gates and valves are complicate, and the external acting devices, with a few millimeters thickness, are too bulky to be used in high-density biochips. The previous solutions to avoid air bubbles are to create a vacuum in the microfluidic system before use. But the method is complicate and not very effective.

As the reaction zones or sensing spots become smaller for high density or multi-analytes array biochip, immobilization of biological probes becomes a challenge. Because the probe area is in a close confinement and as small as 10 μm diameter, it is difficult to avoid cross-contamination for an array of probes needed to be immobilized. Also it is problematic to secure the bioactivity of the immobilized probes, due to multi-layers or multi-thin film assembling processes. More importantly, once the chip is assembled, it is impossible to load or rearrange the probes of interest any more. Therefore, it is preferable to have a simple and flexible method, which can load various biological probes to any desired location of the chip according to use's interest or when the chip is in use. Furthermore, it will be beneficial if a method can provide a programmable or controllable method to immobilize, remove, and replenish new probes in microfluidic channels.

Due to the recent interesting in microfluidic devices, a number of U.S. patents related to the design, manufacture, and method of microfluidics have been issued. U.S. Pat. No. 6,238,538 of Caliper Technologies Corp. discloses a method of using electro-osmotic force to control fluid movement. The channel has a zeta potential to support the electro-osmotic flow to move ionic solutions in the microchannel for analyte electrophoresis separation. U.S. Pat. No. 6,186,660 discloses microfluidic systems incorporating varied channel dimensions. The microfluidic channels have some portion or all of the channels have aspect ratios less than 1.0 to provide uniform flow rates through the channels. The analysis channel, includes a serpentine portion, serves to extend the length of the analyte electrophoresis separation without requiring greater substrate area. U.S. Pat. No. 6,270,641 provides innovative microfluidic geometries to reduce the sample dispersion in turns and junctions of microchannel system. U.S. Pat. No. 6,268,219 discloses a microfluidic apparatus for distributing fluid from a main channel to each of the downstream branch channel with equal pressure. In this embodiment, the microfluidic device has a plurality of layers, each with a respective aperture diameter, and aperture. diameters progressively increasing along the length of the channel. U.S. Pat. No. 4,710,472 illustrates a magnetic separation device for removing magnetic bead-coated cells from a system. Although many patents are related to microfluidic platform, none of the patents teaches solving the problems of air bubble, dead volume, and variable reaction volume encountered in the analysis.

### SUMMARY OF THE INVENTION

As the sample or solution volume being reduced to microliter, picoliter, or nanoliter in the biochip platform, it becomes problematic to deliver an exact amount of reaction volume, either sample or reagent solutions, to the reaction zone. To address this problem, the invention is based on a microfluidic channel with constant cross-section area, and a certain section of the microchannel is immobilized with biological probes. Only a fixed section of the microchannel at the reaction zone with probes could have the opportunity for biochemical reactions. The excessive volume of fluids, which is not at the reaction zone, will not have the chance of sufficient incubation time to react with immobilized probes. The reaction volume, that is equal to the cross-section area of the microchannels multiplied with the length of microchannel immobilized probes, is a constant. Therefore, the apparatus becomes insensitive to excessive volume of sample or reagents.

Another object of this invention is to mitigate the problems associated with air bubble and dead volume in the microchannel. The air bubble or dead volume in the microfluidic channel can easily result in unacceptable error for biological assay or clinical diagnosis. It is known that air bubble forms when a small channel is merged with a large channel or large reaction area, or vice versa. The invention is based on microfluidic channels not only have constant cross-section area, but also have serpent-like structures. The serpent-like structure overlaps with the reaction zone to provide a constant reaction volume. Furthermore, said serpent-like structure has a smooth angle on every turn to avoid accumulation of air bubble or creation of the dead volume. The invention works because of two reasons. First, all the fluids are confined in the channel. There is no sudden increase or decrease in channel size, even at reaction zone; therefore, no air bubble could be generated. Second, all the channels have very smooth and gentle curves so that no dead volume is created.

It is an object of the present invention to provide a simple method to immobilize an array of biological probes in the microfluidic-based apparatus with simplicity and flexibility. In accordance with this aspect of the invention, magnetic beads immobilized with probes can be delivered through the microfluidics into the sensing area. An external magnet generator or source beneath the reaction area is used to trap the magnetic beads with probes. Therefore, probe immobilization can be done after the chip has been assembled.

It is another object of the present invention to provide a controllable method to facilitate a particular type of probes on a particular sensing location, according to the need. The external magnet generator or source has on and off mechanisms; and electronic means for controlling on and off operation of said magnet generator. The apparatus of the present invention may provide a programmable or controllable method to immobilize, remove, and/or replenish new probes. In accordance with this aspect of the invention, a magnetic field can be turned off and the used magnetic beads can be washed or rinsed away.

The present invention has the advantage for high precision bioassay, and the resulting apparatus provides accurate and repeatable results. It should be understood, however, that the detail description and specific examples, while indicating preferred embodiments of the present invention, are given by way of illustration and not of limitation. Further, as is will become apparent to those skilled in the area, the teaching of the present invention can be applied to devices for measuring the concentration of a variety of liquid samples.

### BRIEF DESCRIPTION OF THE DRAWING

FIG. 1 is a perspective view of a biochip with microfluidic channel with constant cross-section area. The bottom plate is immobilized with biological probes in the reaction zone, and the bottom of the top plate has microfluidic channel patterns. The integration of the two plates forms a microfluidic channel in closed confinement.

FIG. 2(a) is the top view of the biochip with a serpent-like microchannel of the present invention.

FIG. 2(b) is the top view of a conventional biochip (prior art) with a small microchannel merges into a much larger reaction area.

FIG. 3 is a perspective view of a fluidic biochip with an array of microfluidic channels, reagent microwells, and controllable magnets, in the chip platform. Various types of probes, either on chip or off chip, can be selectively immobilized on any channels. The immobilization process can be done after the biochip has been assembled.

FIG. 4 shows the programmable and refreshable immobilization probes with the ability to select a particular probe, release, and replenish with new probes in the microfluidic channel.

### DETAILED DESCRIPTION OF EMBODIMENTS OF THE INVENTION

The establishment of miniaturized micro-systems has brought to the immediate horizon the possibility of point-of-care test (POCT) analysis, to be achieved by extremely fast, hand-held, portable laboratories. Today, many firms and research institutes are actively engaged in developing microarray and biochip technologies. Although significant efforts have been put into product development, there is still no highly reliable and reproducible biochip-based POCT commercial product. POCT system should provide simplicity and total automation for clinicians to use. The POCT for clinical chemistry is available.

While its assay protocol, such as I-Stat system, is very simple and is limited to one step reaction based on the change of conductivity. A powerful POCT biochip should have the ability to reliably and accurately automate “multiple steps” reaction. All the current POCT analyzers involving multiple steps reaction have poor sensitivity and reliability in comparison with the large laboratory systems. The key problem associated with multiple steps reaction is the variation in each step of sample or reagent delivery. Especially, they are occurred in closed confinement. For example, a common sandwiched immunoassay, three to six reaction steps are required depends on the assay protocol and washing process. Reagent solutions include buffer solutions, antibody conjugates, and labeling materials. Washing steps may be necessary after each reaction. The chip may contain patented microwells and microchannels. All reagent wells may be connected to a reaction zone.

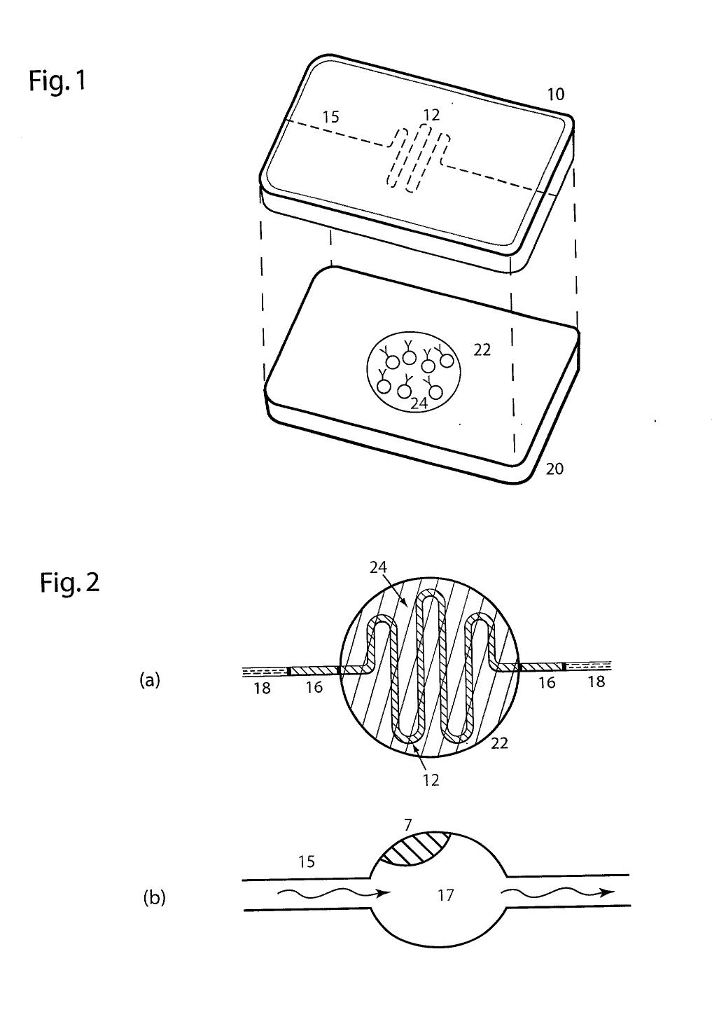

The biochip is designed for whole blood, serum, plasma, urine, and other biological fluid applications. The present invention can be applied to self-contained biochip and high throughput biochip. For a self-contained biochip, a predetermined amount of the reagents are stored in pre-packaged sealed plastic pouches or microwells. For a high throughput biochip, because large amount of reagents are needed, the reagents are provided from external reservoirs. FIG. 1 shows the major portion of the biochip with microchannel in the reaction zone. The biochip platform may be made of plastic, glass, silicon, or other hybrid materials. The common plastic chip materials used are polystyrene, polycarbonate, polydimethylsiloxane (PDMS), or polypropylene. It is preferable to be optically clear. The top surface of a base plate 20 is immobilized with biological probes 24 on the reaction zone 22. The reaction zone is the area where the probes are immobilized.

The probes have the ability to react with analytes of interest in the media. The probes can be biological cells, proteins, antibodies, antigens, nucleic acids, enzymes, or other biological receptors. Antibodies are used to react with antigens. Oligonucleotides are used to react with the complementary strain of nucleic acid. There are many immobilization methods including physical and chemical attachments; they are well known to those who are skilled in the art. Polystyrene-based microplate is most commonly used to physically bind probes on the surface. Various chemical adsorption methods, such as biotinylated antibody to the activated surface (treated with hydroxysuccinimide), are used to ensure long-term stability. To minimize non-specific binding, a blocking solution (e.g. BSA, Tween 20) can be applied to the surface after the probe immobilization.

The top plate 10 is a thin layer of plastic material. At the bottom of the top plate is patterned with microfluidic circuitry 15. The microfluidic circuitry is designed according to assay protocol. The two plates are coupled and formed microchannels in closed confinement. In this invention, the microfluidic channel has a fixed cross-section area from the inlet section, at the sample or reagent storage sections, to the end section of the channel, at waste reservoirs. A section of microchannel with serpent-like structure 12 overlay on top of the probes immobilized on the reaction zone 22. The serpent-like structure 12 is also commonly known as “curved structure”. The dimension of the typically microchannel or microfluidic channel is between 0.5 μm to 2 mm equivalent diameter, wherein the “equivalent diameter” is defined as the square root of “the area divided by π”. As illustrated, the top plate 10 contains one layer however, one skilled in the art would recognize that the top plate may comprise several layers.

FIG. 2(a) is the top view of the biochip with a serpent-like microchannel 12 overlaid on top of the biological probes immobilized on the reaction zone 114. The reaction volume 14 is defined as the cross-section area of the microfluidic channel multiplied with the length of the channel in the reaction zone. Because the cross-section area and the length are fixed, thus the total reaction volume is a constant number regardless how much sample or reagent is delivered. Only the solution “inside the reaction zone” 22 has chance to react with the probes 24. For example, it does not matter if we deliver 12 μl or 15 μl of sample to the sample port, only 10 μl (constant reaction volume) of solution would react with the immobilized probes in the reaction zone.

By design, the sample and reagent volumes are more than the reaction volume. Therefore, the excessive solution 16, which is outside the reaction zone 22, does not have the opportunity or sufficient incubation time to react with immobilized probes. A series of reagent solutions 18 are sequentially transported through and incubated at the reaction zone for reactions. The probe-target reactions can be monitored with an optical detector located above the reaction zone. Optical detectors are used to detect the change of absorption, fluorescence, light scattering, and chemiluminescence due the probe-target reactions. The fluid in microfluidic channels is moved by a pressurizing mechanism, such as pumping, micro-actuator, electro-osmotic force, acoustic wave, voltage gradient, and capillary action, for providing a forward-moving fluid. The typical reaction volume ranges from 0.5-500 μl. This structure of the present biochip design makes it insensitive to the sample or reagent volumes.

It is commonly observed that air bubbles or dead volumes are easily formed in microfluidic systems. Especially, as shown in FIG. 2(b), when a solution is transported from a small channel 15 into a large reaction area 17. Due to non-uniform fluid distribution in the large reaction zone, dead volume 7 might be created. The improper shape or curve (e.g. 90 degree turn) of the microfluidic channel could also cause air bubbles to accumulate at the corner of the channel. The serpent-like microfluidic channel not only has the same cross-section area, even at the reaction zone, but also a gently curved structure. There is no sudden increase or decrease in channel dimension. The fluids continuously move from sample well or reagent well to the reaction area, and then to waste reservoir in the microchannel. There is little chance to form a dead volume or air bubbles.

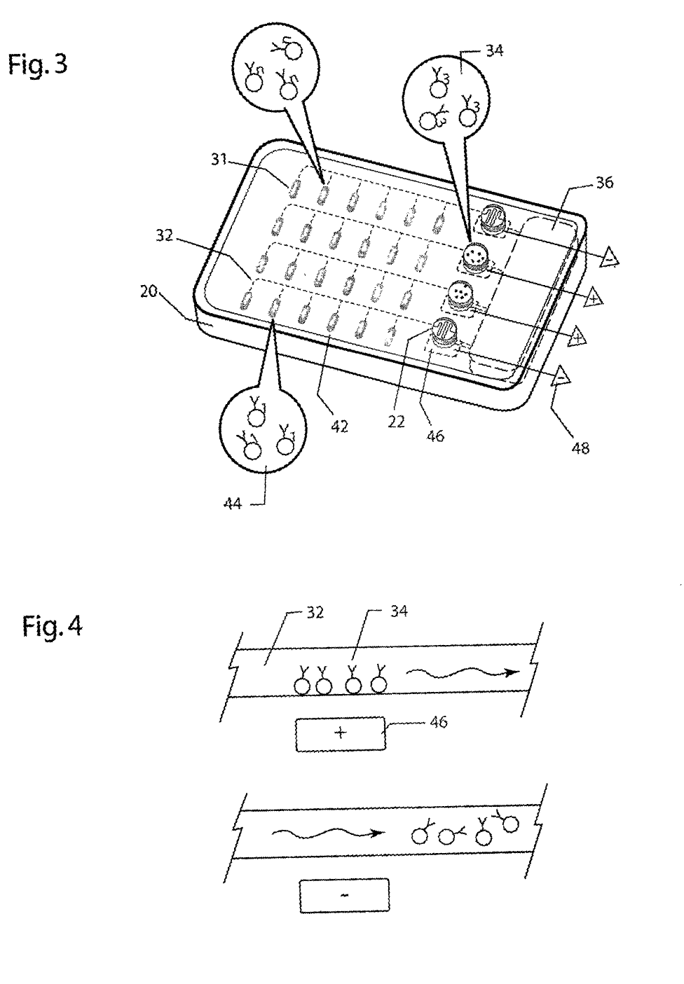

The biochip, as shown in FIG. 3, is constructed with many micro-cavities or microwells for loading the samples 31 and necessary reagents 42 for the reaction. The microwells are connected to the microfluidic network 32 and a reaction zone 22. The probes immobilization at the reaction zone is one embodiment of this invention.

There are two methods for probe immobilization. The first method is to directly immobilize the probes on the surface of plate at the reaction zone. This method is easy for immobilization on the bottom of a microwell, but is difficult on planar biochip platform. Especially, multilayers of thin films or plates need to be adhered together. It has been appreciated if the immobilization process in the microfluidic biochip can be done with much simpler and flexible method. The typical microwell size in microplates is 8 mm in diameter and 10 mm in depth. Other suitable dimensions may equally be applicable.

The second method is to use magnetic beads as a supporting material for probe immobilization. Magnetic beads or particles are used widely for separating, purifying, and analyzing biological analytes. Magnetic bead is one of the most successful solid supports to bind the probe molecules. The utilization of magnetic beads, such as latex bead, has been developed for more than 40 years. Magnetic beads are commonly used to immobilize the probes on the bottom of the microwell or microtiter plate for solid phase immunoassay or ELISA. Tremendous efforts were gone into developing high yield and high stability polymer coating and surface functional groups for target-probe chemistry. However, all current immobilization processes are performed in free space with apparent disadvantages for biochip application.

This invention provides a simple, retrofitted, flexible, and controllable probe immobilization method. FIG. 3 shows a biochip combining a microfluidic channel 32, probe coated magnetic particles 44, an array of magnets 46, and control electronics 48 to provide a unique immobilization method. The apparatus offers simplicity to facilitate various probes on the microfluidic platform. The invention offers the advantage of probe-in-demand flexibility. It means that the users can select their own set of probes for analytes testing.

An array of magnets 46 installed beneath an array of microfluidic channels. The magnet source or generator is controlled by electronics 48 with the ability to activate and deactivate a particular magnet electronically, when the magnetic beads 44 are transported from the storage microwell through the microfluidic channels. If the magnet source is not turned on, the magnetic beads 44 flow through the system to the waste reservoir 36. However, if a particular magnet or magnet source is activated, the magnetic beads could be trapped by the magnetic field in the reaction zone as immobilized probes 34. Various probes can be coated on the magnetic beads for a series of reactions. The dimension of a magnetic bead is typically between about 50 nm to about 300 μm.

FIG. 4 shows the cross section of the microfluidic system with magnetic beads 34 in the microfluidic channel, and magnets 46 installed under the channel 32. If the beads are immobilized with a particular type of probe (e.g. antibody), when the specific target (e.g. antigen) is flown through the probes in the channel, the antigen molecules will be reacted and caught by the antibody immobilized on the magnetic beads 34. By this method, this apparatus provides a simple and flexible method to facilitate a particular type of probes on a particular sensing need.

The present invention also provides external magnet sources, which have on and off mechanisms; and electronic means 48 for controlling on and off of said magnet. The resulting apparatus provides a programmable or digitally controllable method to immobilize, remove, and replenish new probes. In accordance with this aspect of the invention, a magnetic field can be turned off and the used magnetic beads can be washed away.

The claim of the invention is:

1. A method for performing biological assay in a microfluidic biochip platform providing constant and consistent reaction volume defining a reaction zone, the method comprising the steps of:
(a) providing a plurality of microfluidic channels with a constant cross-section area;
(b) immobilizing at least one biological probe on said reaction zone; and
(c) transporting fluid in said microfluidic channels to said reaction zone, a portion of said fluid reacting with said at least one probe, wherein said reaction volume is product of said cross-section area multiplied with length of said microfluidic channels having said at least one biological probe.

2. The method as defined in claim 1, wherein a portion of said microfluidic channels has serpent-like structure, said serpent-like structure overlaying with at least a portion of said reaction zone.

3. The method as defined in claim 1 or 2, wherein said microfluidic channels have dimension between 0.5 μm and 2 mm in cross-section.

4. The method as defined in claim 1 or 2, the microfluidic biochip platform further comprising at least one sample source and at least one reagent solution, wherein a portion of said microfluidic channels is connected to said at least one sample source and to said at least one reagent solution.

5. The method defined in claim 1 or 2, wherein said fluid in said microfluidic channels is moved by a pressurizing mechanism for providing a forward-moving fluid.

6. The method defined in claim 1 or 2, the method further comprising the steps of:
(a) immobilizing said at least one biological probe on magnetic beads;
(b) transporting said magnetic beads through said microfluidic channels;
(c) providing at least one external magnet from magnet sources beneath said reaction zone; and
(d) switching on said at least one external magnet to trap said magnetic beads.

7. The method defined in claim 2, wherein said biochip platform further comprises:
(a) said at least one biological probe immobilized on said reaction zone of a base plate;
(b) said microfluidic channels patterned on a bottom surface of a top plate; and
(c) said top plate coupled on top of said base plate.

8. The microfluidic biochip platform according to claim 1 or 2, wherein said probe is protein.

9. The microfluidic biochip platform according to claim 1 or 2, wherein said probe is nucleic acid.

10. The microfluidic biochip platform according to claim 1 or 2, wherein said probe is biological cell.

11. The microfluidic biochip platform according to claim 1 or 2 further comprising an optical detector located above said reaction zone.

12. A method for performing biological assay in a biochip with an array of microfluidic channels providing flexible and controllable immobilization for at least one biological probe, the method comprising the steps of:
(a) immobilizing said at least one biological probe on magnetic beads;
(b) selecting at least one of said magnetic beads and transporting said magnetic beads through one of said microfluidic channels;
(c) providing at least one external magnet beneath a portion of said microfluidic channels; and
(d) switching on said at least one external magnet for immobilization of at least one of said at least one biological probe.

13. The method defined in claim 12, wherein said external magnets have on and off switching mechanisms for immobilizing or removing said biological probe in said microfluidic channels; and an electronic means for controlling said on and off switching mechanisms.

14. The method as defined in claim 12, wherein said microfluidic channels have dimension between 0.5 μm and 2 mm in cross-section.

15. The method as defined in claim 12, the biochip further comprising at least one sample source and at least one reagent solution, wherein a portion of said microfluidic channels is connected to said at least one sample source and to said at least one reagent solution.

16. The method defined in claim 12, wherein said fluid in said microfluidic channels is moved by a pressurizing mechanism for providing a forward-moving fluid.

17. The biochip according to claim 12, wherein said probe is protein.

18. The biochip according to claim 12, wherein said probe is nucleic acid.

19. The biochip according to claim 12, wherein said probe is biological cell.

20. The biochip according to claim 12 further comprising an optical detector located above said microfluidic channels.
